(19) United States
(12) Patent Application Publication
(10) Pub. No.: US 2017/0273732 A1
(43) Pub. Date: Sep. 28, 2017

(54) REGIONAL FLOW SENSOR ON CARDIAC CATHETER

(71) Applicant: Boston Scientific Scimed Inc., Maple Grove, MN (US)

(72) Inventors: Sarah R. Gutbrod, St. Paul, MN (US); Mary M. Byron, Roseville, MN (US); Jacob I. Laughner, St. Paul, MN (US); Jason J. Hamann, Blaine, MN (US)

(21) Appl. No.: 15/467,837

(22) Filed: Mar. 23, 2017

Related U.S. Application Data
(60) Provisional application No. 62/312,725, filed on Mar. 24, 2016.

(51) Int. Cl. A61B 18/08; A61B 18/12

(52) U.S. Cl. CPC A61B 18/08; A61B 18/1206; A61B 2018/00351

(57) ABSTRACT

Catheters and methods are provided for determining a fluid flow rate of fluid located near a tip assembly of the catheter and providing an indication of the fluid flow condition and/or controlling one or more functions of the catheter based on the determined fluid rate. A method includes receiving a signal from a sensor located on a tip assembly of a catheter, the signal being indicative of a fluid flow condition of fluid located near the tip assembly. A processing device determines the fluid flow condition using the signal. Based on the determined fluid flow rate, the processor causes a device to output an indication of the determined fluid flow condition.

### CROSS-REFERENCE TO RELATED APPLICATION

This application claims priority to Provisional Application No. 62/312,725, filed Mar. 24, 2016, which is herein incorporated by reference in its entirety.

### TECHNICAL FIELD

Embodiments of the present disclosure relate to medical devices. More specifically, embodiments of the disclosure relate to catheters and methods for determining a fluid flow condition of fluid located near a tip assembly of the catheter and providing an indication of the fluid flow condition and/or controlling one or more functions of the catheter based on the fluid flow condition.

### BACKGROUND

Cardiac arrhythmias involve irregularities in the transmission of electrical impulses through the heart. To treat cardiac arrhythmias, physicians often use mapping and ablation catheters to map cardiac tissue and create ablation lesions on tissue that may be contributing to the arrhythmia (referred to herein as “target tissue”). The ablation lesions, when properly formed on the target tissue, physiologically alter the target tissue by disrupting and/or blocking electrical pathways through the target tissue. In addition to sufficiently disrupting and/or blocking electrical pathways through the target tissue, it is important not to disrupt or block electrical activity that is conducted through healthy tissue surrounding the target tissue. In order to effectively isolate the target tissue, the flow of blood and irrigation fluid, if an irrigated catheter is being used, near the target tissue may be taken into account. If the fluid flow condition is not considered, ineffective passive cooling, charring and steam popping, may occur during ablation. Furthermore, if the fluid flow condition is not considered, a larger or smaller portion of tissue may be ablated than was intended.

### SUMMARY

Embodiments of the disclosure relate to catheters and methods for determining a fluid flow condition of fluid located near a tip assembly of the catheter and providing an indication of the fluid flow condition and/or controlling one or more functions of the catheter based on the determined fluid flow condition.

In Example 1, a method comprises: receiving a signal from at least one sensor located on a tip assembly of a catheter, the signal being indicative of a fluid flow condition of fluid located near the tip assembly; determining the fluid flow condition using the signal; and causing a device to output an indication of the determined fluid flow condition.

In Example 2, the method of Example 1, wherein the fluid flow condition includes at least one of: a stagnant flow condition, a low flow condition and a high flow condition.

In Example 3, the method of any of Examples 1-2, wherein the fluid flow condition includes a fluid flow rate.

In Example 4, the method of Example 3, further comprising providing a notification to a user when the determined fluid flow rate is below a fluid flow rate threshold.

In Example 5, the method of any of Examples 3-4, wherein the device is a display device, the method further comprising causing the display device to display a representation of the determined fluid flow rate.

In Example 6, the method of Example 5, further comprising: displaying, using the display device, a representation of a cardiac structure; and displaying a representation of the determined fluid flow rate on the representation of the cardiac structure.

In Example 7, the method of any of Examples 1-4, wherein the device is a sensory output device incorporated into a handle of the catheter and wherein the indication of the determined fluid flow condition is at least one of: one or more colors of light, one or more sequences of lights, one or more sounds, one or more sequence of sounds and one or more haptics.

In Example 8, the method of any of Examples 1-7, wherein tip assembly includes a conductive exterior wall for delivering RF energy for an RF ablation procedure and an irrigation port, the method further comprising at least one of: controlling an amount of irrigation fluid provided to the irrigation port based on the determined fluid flow rate and controlling an amount of RF energy delivered by the conductive exterior wall based on the determined fluid flow condition.

In Example 9, the method of any of Examples 1-8, wherein the catheter is an irrigated catheter, the method further comprising determining an irrigation flow condition of irrigation fluid using the determined fluid flow condition.

In Example 10, the method of any of Examples 1-9, the at least one flow sensor including at least one of the following: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor and an ionic concentration sensor.

In Example 11, a catheter system comprises: a catheter comprising a tip assembly, the tip assembly including: a conductive exterior wall for delivering radio frequency (RF) energy for an RF ablation procedure, an irrigation port and at least one sensor; and a processing device configured to: receive a signal from the at least one sensor, the signal being indicative of a fluid flow rate of fluid located near the tip assembly; determine the fluid flow rate using the signal; and control, based on the determined fluid flow rate, at least one of an amount of delivered RF energy and an amount of irrigation fluid provided to the irrigation port.

In Example 12, the system of any of Examples 11-12, the processing device further configured to determine when the determined fluid flow rate is below a fluid flow rate threshold and wherein controlling at least one of an amount of delivered RF energy and an amount of irrigation fluid provided to the irrigation port includes at least one of: increasing the amount of irrigation fluid provided to the irrigation port and decreasing the amount of delivered RF energy, when the determined fluid flow rate is below the fluid flow rate threshold.

In Example 13, the system of any of Examples 11-12, the processing device being further configured to provide a notification to a user when the determined fluid flow rate is below a fluid flow rate threshold.

In Example 14, the system of any of Examples 11-13, the processing device being further configured to cause a display device to display a representation of the determined fluid flow rate.

In Example 15, the system of any of Examples 11-14, the at least one flow sensor including at least one of the following: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor and an ionic concentration sensor.

In Example 16, a catheter system comprises: a catheter comprising a tip assembly, the tip assembly including at least one sensor; and a processing device configured to: receive a signal from the at least one sensor, the signal being indicative of a fluid flow condition of fluid located near the tip assembly; determine the fluid flow condition using the signal; and cause a device to output an indication of the determined fluid flow condition.

In Example 17, the system of Example 16, wherein the fluid flow condition includes at least one of: a stagnant flow condition, a low flow condition and a high flow condition.

In Example 18, the system of Example 16, wherein the fluid flow condition includes a fluid flow rate.

In Example 19, the system of Example 18, the processing device being further configured to provide a notification to a user when the determined fluid flow rate is below a fluid flow rate threshold.

In Example 20, the system of Example 18, the processing device being further configured to cause the display device to display a representation of the determined fluid flow rate.

In Example 21, the system of Example 19, the processing device being further configured to cause the display device to display the representation of the determined fluid flow rate on a representation of a cardiac structure.

In Example 22, the system of Example 16, wherein the device is a sensory output device incorporated into a handle of the catheter and wherein the indication of the determined fluid flow condition is represented by at least one of: one or more colors of light, one or more sequences of lights, one or more sounds, one or more sequence of sounds and one or more haptics.

In Example 23, the system of Example 16, wherein tip assembly includes a conductive exterior wall for delivering RF energy for an RF ablation procedure and an irrigation port, the processing device being further configured to control at least one of: an amount of irrigation fluid provided to the irrigation port and an amount of RF energy delivered by the conductive exterior wall, based on the determined fluid flow condition.

In Example 24, the system of Example 23, wherein the fluid flow condition includes a fluid flow rate and wherein to control an amount of irrigation fluid provided to the irrigation port based on the determined fluid flow rate, the processing device is configured to increase the amount of irrigation fluid provided to an irrigation port when the determined fluid flow rate is below a fluid flow rate threshold.

In Example 25, the system of Example 16, wherein the catheter is an irrigated catheter, the processing device being further configured to determine an irrigation flow condition of irrigation fluid using the determined fluid flow condition.

In Example 26, the system of Example 16, the at least one flow sensor including at least one of the following: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor and an ionic concentration sensor.

In Example 27, a method comprises: receiving a signal from at least one sensor located on a tip assembly of a radio frequency (RF) ablation catheter, the signal being indicative of a fluid flow condition of fluid located near the tip assembly; determining the fluid flow condition using the signal; and dynamically adjusting, based on the determined fluid flow rate, at least one of an amount of RF energy delivered by the tip assembly and an amount of irrigation fluid provided to an irrigation port included in the tip assembly.

In Example 28, the method of Example 27, further comprising determining an irrigation flow rate using the determined fluid flow rate and providing a notification to a user when the determined fluid flow rate is below a fluid flow rate threshold.

In Example 29, the method of Example 27, further comprising causing a display device to display a representation of the determined fluid flow rate.

In Example 30, the method of Example 27, further comprising: displaying, using a display device, a representation of a cardiac structure; and displaying a representation of the determined fluid flow rate on the representation of the cardiac structure.

In Example 31, the method of Example 27, the at least one flow sensor including at least one of the following: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor and an ionic concentration sensor.

In Example 32, a catheter system comprises: a catheter comprising a tip assembly, the tip assembly including: a conductive exterior wall for delivering radio frequency (RF) energy for an RF ablation procedure, an irrigation port and at least one sensor; and a processing device configured to: receive a signal from the at least one sensor, the signal being indicative of a fluid flow rate of fluid located near the tip assembly; determine the fluid flow rate using the signal; and control, based on the determined fluid flow rate, at least one of an amount of delivered RF energy and an amount of irrigation fluid provided to the irrigation port.

In Example 33, the system of Example 32, the processing device being further configured to cause the display device to display the representation of the determined fluid flow rate on a representation of a cardiac structure.

In Example 34, the system of Example 32, the processing device being further configured to determine an irrigation flow rate of irrigation fluid exiting the irrigation port using the determined fluid flow rate.

In Example 35, the system of Example 32, the at least one flow sensor including at least one of the following: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor and an ionic concentration sensor.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 depicts an illustrative mapping and ablation system that includes a catheter having a sensor that senses signals indicative of a fluid flow condition and provides an indication of the fluid flow condition and/or controls the catheter based on the fluid flow condition, in accordance with embodiments of the disclosure.

FIGS. 2A-2D depict illustrative catheter tip assemblies, in accordance with embodiments of the disclosure.

FIG. 3 depicts a representation of fluid flow conditions on a representation of a cardiac structure, in accordance with embodiments of the disclosure.

FIG. 4 is a schematic block diagram of an illustrative process for determining a fluid flow condition and providing an indication of the fluid flow condition and/or controlling a catheter based on the determined fluid flow condition, in accordance with embodiments of the disclosure.

FIG. 5 is a flow diagram depicting an illustrative method for determining a fluid flow condition and providing an indication of the fluid flow condition and/or controlling a catheter based on the determined fluid flow condition, in accordance with embodiments of the disclosure.

While the disclosed subject matter is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

As the terms are used herein with respect to ranges of measurements (such as those disclosed immediately above), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

Although the term “block” may be used herein to connote different elements illustratively employed, the term should not be interpreted as implying any requirement of, or particular order among or between, various steps disclosed herein unless and except when explicitly referring to the order of individual steps.

### DETAILED DESCRIPTION

Embodiments of this disclosure relate to catheters and methods for determining a fluid flow condition of fluid located near a tip assembly of a catheter and providing an indication of the fluid flow condition and/or controlling one or more functions of the catheter based on the determined fluid flow condition. In embodiments, the fluid flow condition may be indicative of a fluid flow level (e.g., stagnant flow level, low flow level and/or high flow level), a fluid flow rate, a fluid flow velocity, a fluid flow acceleration (with or without a direction) and/or the like. As such, embodiments of this disclosure may facilitate reducing the likelihood of an adverse event occurring during an ablation procedure.

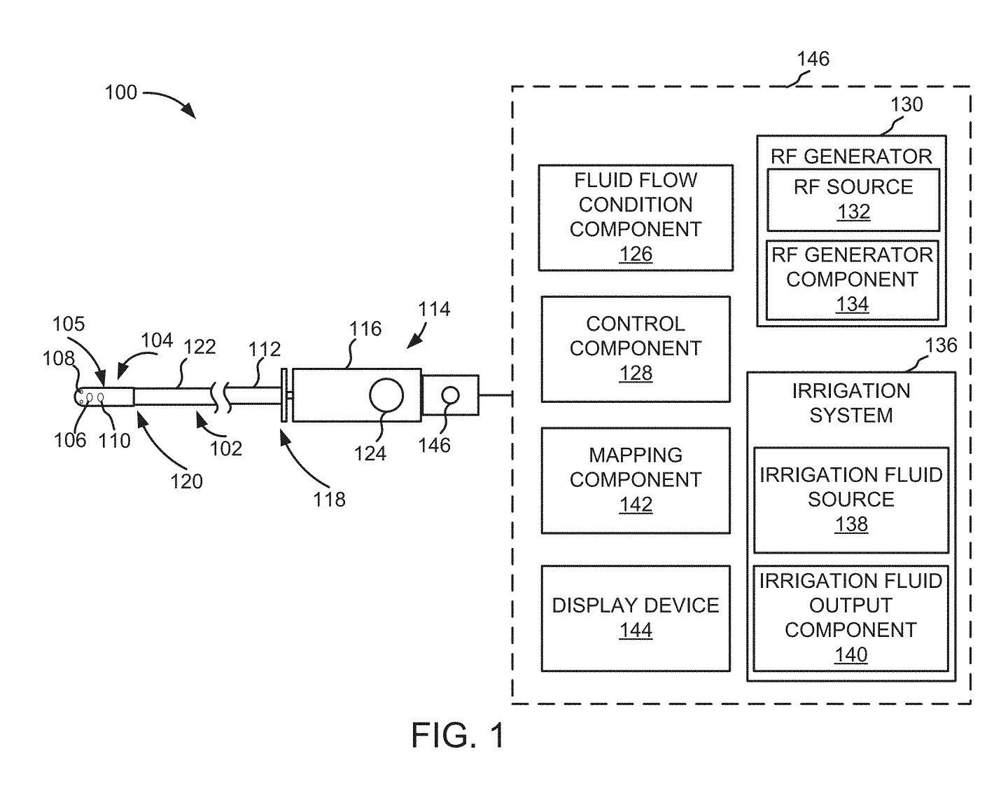

FIG. 1 depicts a mapping and ablation system 100 that includes an open-irrigated ablation catheter 102, according to embodiments of the disclosure. While an open-irrigated ablation catheter 102 is depicted, other catheters, for example, a diagnostic catheter, a mapping catheter, a sheath catheter and/or a non-irrigated ablation catheter, may be used in the embodiments described herein. The illustrated catheter 102 includes a tip assembly 104 having a tissue ablation electrode 105, with mapping electrodes 106, distal irrigation ports 108 and at least one sensor 110. In embodiments, the catheter 102 may be a closed-irrigated catheter or a non-irrigated catheter. The catheter 102 includes a catheter body 112 and a proximal catheter handle assembly 114, having a handle 116, coupled to a proximal end 118 of the catheter body 110. The tip assembly 104 is coupled to a distal end 120 of the catheter body 110.

In embodiments, the mapping and ablation system 100 may be utilized in ablation procedures on a patient and/or in ablation procedures on other objects. In various embodiments, the ablation catheter 102 may be configured to be introduced into or through the vasculature of a patient and/or into or through any other lumen or cavity. In an example, the ablation catheter 102 may be inserted through the vasculature of the patient and into one or more chambers of the patient's heart (e.g., a target area). When in the patient's vasculature or heart, the ablation catheter 102 may be used to map and/or ablate myocardial tissue using the electrodes 106 and/or the tissue ablation electrode 105. In embodiments, the tissue ablation electrode 105 may be configured to apply ablation energy to myocardial tissue of the heart of a patient. Furthermore, in embodiments, the sensor 110 senses signals indicative of a fluid flow condition of fluid located near the tip assembly 104. Based on the fluid flow condition, the catheter 102 may be controlled in a manner described herein.

According to embodiments, the tissue ablation electrode 105 may be, or be similar to, any number of different tissue ablation electrodes such as, for example, the IntellaTip MiFi,™ Orion™ or the Blazer™ Ablation tip, all of which are available from Boston Scientific of Marlborough, Massachusetts. In embodiments, the tissue ablation electrode 105 may have any number of different sizes, shapes, and/or other configuration characteristics. The tissue ablation electrode 105 may be any length and may have any number of the electrodes 106 positioned therein and spaced circumferentially and/or longitudinally about the tissue ablation electrode 105. In some instances, the tissue ablation electrode 105 may have a length of between one (1) mm and twenty (20) mm, three (3) mm and seventeen (17) mm, or six (6) mm and fourteen (14) mm. In an illustrative example, the tissue ablation electrode 105 may have an axial length of about eight (8) mm. In another illustrative example, the tissue ablation electrode 105 may include an overall length of approximately 4-10 mm. In embodiments, the tissue ablation electrode 105 may include an overall length of approximately 4 mm, 4.5 mm, and/or any other desirable length. In some cases, the plurality of electrodes 106 may be spaced at any interval about the circumference of the tissue ablation electrode 105. In an example, the tissue ablation electrode 105 may include at least three electrodes 106 equally or otherwise spaced about the circumference of the tissue ablation electrode 105 and at the same or different longitudinal positions along the longitudinal axis of the tissue ablation electrode 105.

In embodiments, the electrodes 106 may be configured to operate in unipolar or bipolar sensing modes. In embodiments, the electrodes 106 may define and/or at least partially form one or more bipolar electrode pairs, each bipolar electrode pair being configured to measure an electrical signal corresponding to a sensed electrical activity (e.g., an electrogram (EGM) reading) of the myocardial tissue proximate thereto. The sensed signals from the electrodes 106 may be provided to the mapping component 142 for processing as described herein. In embodiments, an EGM reading or signal from a bipolar electrode pair may at least partially form the basis of a contact assessment, ablation area assessment (e.g., tissue viability assessment), and/or an ablation progress assessment (e.g., a lesion formation/maturation analysis), as discussed below.

In embodiments, the distal tip 104 includes irrigation ports 108. In embodiments, the irrigation ports 108 may contribute to reducing coagulation of blood near the tip assembly 104. For example, when the tissue ablation electrode 105 is applying ablation energy to cardiac tissue, an irrigation system 136, as described herein, may provide cooling fluid, such as a saline, through the catheter 102 and out through the irrigation ports 108 in order to cool the blood. In embodiments, the amount of irrigation fluid provided to an irrigation port 108 may be based on a determined fluid flow condition as described herein.

In embodiments, the distal tip 104 includes at least one sensor 110. When the ablation catheter 102 is in the patient's vasculature or heart, the sensor 110 is configured to sense signals indicative of a fluid flow condition. In embodiments, the fluid flow condition may be the fluid flow condition of blood (and/or other bodily fluids) near the tip assembly 104, the fluid flow condition of irrigation fluid near the tip assembly 104 and/or a combination of the blood and irrigation fluid near the tip assembly 104. To sense a fluid flow condition, one or more of the following sensors may be used: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor, an ionic concentration sensor, and/or the like. Embodiments of these sensors are described in more detail below. The sensed signals indicative of the fluid flow condition may be provided to a fluid flow condition component 126 for determining a fluid flow condition based on the signals as described herein.

In embodiments, the catheter 102 may include a deflectable catheter region 122 configured to allow the catheter 102 to be steered through the vasculature of a patient, and which may enable the tissue ablation electrode 105 to be accurately placed adjacent a targeted tissue region. A steering wire (not shown) may be slidably disposed within the catheter body 110. The handle assembly 114 may include one or more steering members 124 such as, for example, rotating steering knobs that are rotatably mounted to the handle 116. Rotational movement of a steering knob 124 relative to the handle 116 in a first direction may cause a steering wire to move proximally relative to the catheter body 112 which, in turn, may tension the steering wire, thus pulling and bending the catheter deflectable region 122 into an arc; and rotational movement of the steering knob 124 relative to the handle 116 in a second direction may cause the steering wire to move distally relative to the catheter body 110 which, in turn, may relax the steering wire, thus allowing the catheter 102 to return toward its original form. To assist in the deflection of the catheter 102, the deflectable catheter region 122 may be made of a lower durometer plastic than the remainder of the catheter body 110.

According to embodiments, the catheter body 110 includes one or more cooling fluid lumens (not shown) to provide cooling fluid to an irrigation port 108 and may include other tubular element(s) to provide desired functionality to the catheter 102. The addition of metal in the form of a braided mesh layer sandwiched in between layers of plastic tubing may be used to increase the rotational stiffness of the catheter 102.

As mentioned above, the signals sensed by the sensor 110 are provided to the fluid flow condition component 126. In embodiments, the fluid flow condition component 126 determines a fluid flow condition of fluid located near the tip assembly 104 based on the signals received from the sensor 110. In embodiments, the fluid flow condition may be indicative of a fluid flow level (e.g., stagnant flow level, low flow level and/or high flow level), a fluid flow rate, a fluid flow velocity, a fluid flow acceleration (with or without a direction) and/or the like. In embodiments, a stagnant flow may be a fluid flow level that is non-existent or minimal. In embodiments, a low flow level may be a fluid flow level that is typically measured in the appendages/conduits of a heart. In embodiments, a high flow level may be a fluid flow level that is typically measured near valves of the heart. In embodiments, the determination of the fluid flow condition by the fluid flow condition component 126 may depend on the type of sensor 110, as described below. In embodiments, the fluid flow condition component 126 may also determine an irrigation fluid flow condition. The fluid flow condition component 126 may determine an irrigation fluid flow condition by measuring a fluid flow condition before an irrigation system (e.g., the irrigation system 136) is turned on and again, after the irrigation system is turned on. If the tip assembly 104 has not moved, an irrigation fluid flow condition may be determined by subtracting the fluid flow conditions before and after the irrigation system was turned on.

For example, in embodiments where the sensor 110 is a thermal flow sensor, the sensor 110 can include two components, a heat source and a temperature probe, that are surface mounted to the tip assembly 104. The heat source emits a known amount of heat and the temperature sensor measures an amount of heat. The fluid flow condition component 126 may receive the emitted and measured heat signals in order to determine a difference between the two signals. The difference between the two signals is indicative of a temperature decrease. In embodiments, the fluid flow condition component 126 correlates the temperature decrease to one of a plurality of fluid flow levels and/or fluid flow rates to determine the fluid flow condition of fluid near the tip assembly 104.

As another example, in embodiments where the sensor 110 is a pressure sensor, the sensor 110 may include a window in the distal tip 104 that allows fluid to flow through the window and a component located internal to the window. The window and the component located internal to the window each have sensors that can sense a pressure. In embodiments, the fluid flow condition component 126 receives a signal indicative of the pressure that is applied to the window by the fluid. And, the fluid flow condition component 126 receives a signal indicative of the pressure that is applied to the component. The difference between the two signals can be correlated to a fluid flow level and/or fluid flow rate by the fluid flow condition component 126 to determine a fluid flow level and/or fluid flow rate of fluid located near the distal tip 104. In addition, or alternatively, the sensor 110 may include one or more pressure sensors that sense one or more pressure gradients. From the pressure gradients, the fluid flow condition component 126 may determine a direction of the fluid flow.

As another example, in embodiments where the sensor 110 is an optical and/or an ultrasonic sensor, the sensor 110 may emit an optical and/or sound signal and receive a reflected signal of the emitted signal. The reflected signal may be scattered by one or more particles located in the blood and/or cardiac tissue of the patient. The reflected and emitted signals are subject to a Doppler shift, which can be correlated to one of a plurality of fluid flow levels and/or fluid flow rates by the fluid flow condition component 126.

As another example, in embodiments where the sensor 110 is a pH sensor and/or an ionic concentration sensor, the sensor 110 may include a permeable membrane that coats an electrode in Hydrogen ions, potassium and/or sodium. By measuring the voltage differential between the coated sensor and a reference electrode, the pH and/or ionic concentration of the fluid can be determined. In embodiments, the reference electrode may be a separate electrode and/or may be an electrode that is also used as a mapping electrode. In embodiments, the oscillations in the measurements of the pH and/or ionic concentration can be correlated to the fluid flow level and/or the fluid flow rate by the fluid flow condition component 126. In embodiments, the measured pH and/or ionic concentration may depend on temperature. As such, a temperature sensor may be incorporated into the sensor 110 to sense temperatures, which can be used by the fluid flow condition component 126 to normalize the measured pH and/or ionic concentration.

As another example, in embodiments where the sensor 110 includes an oxygen sensor, the sensor 110 may sense oxygen and determine an amount of luminescence in the sensed oxygen. The determined luminescence in the oxygen can be correlated to one of a plurality of fluid flow levels and/or fluid flow rates by the fluid flow condition component 126. In embodiments, the luminescence measurements may depend on conductivity. As such, an impedance sensor may be incorporated into the sensor 110 and used by the fluid flow condition component 126 to normalize the measured luminescence.

As another example, in embodiments where the sensor 110 includes a mechanical sensor (e.g., a volumetric sensor, a linear pair sensor and/or a concentric pair sensor), the sensor 110 may include a membrane and/or appendage that can be deflected by the fluid surrounding the tip assembly 104. The degree of deflection and orientation of deflection of the membrane and/or appendage is measured by one or more strain gauges included in the sensor 110. The displacement vector of the one or more strain gauges can then be correlated to one of a plurality of fluid flow levels and/or fluid flow rates by the fluid flow condition component 126.

Furthermore, in embodiments, one or more of the plurality of sensors described above may be used to measure in combination with one another. For example, a second sensor of the plurality sensors may be used to verify a fluid flow condition that was determined using a first sensor of the plurality of sensor. As another example, a fluid flow condition may be determined by taking a weighted average of one or more of the plurality of sensors. For example, in a weighted average computation, the thermal fluid flow sensor and/or the mechanical sensor may be weighted the most heavily, followed by the optical sensor, the pH sensor and the oxygen sensor, in that order. As even another example, the pH and the pressure sensor may be used in conjunction. For example, the pressure sensor may be linearly dependent on the pH. As such, the pressure sensor may be adjusted linearly, depending on the pH level of the fluid.

The examples given above are not meant to be limiting. Instead, any type of sensor that can be used to determine a fluid flow condition may be used as the sensor 110. Furthermore, any combination of sensors 110 may be used in combination to verify one another and/or provide a more accurate fluid flow condition determination.

The illustrated system 100 includes a control component 128. The control component 128 may be configured to control aspects of the functioning of one or more other components such as, for example, an RF generator 130, an irrigation system 136 (described in further detail below), and/or the like. In embodiments, the control component 128 may be configured to adjust one or more operation parameters based on information such as, for example, user input, input from other components (e.g., the fluid flow condition component 126), and/or the like.

In embodiments, for example, the control component 128 may receive the fluid flow condition determined by the fluid flow condition component 126 and control an RF generator 130. The RF generator 130 included in the system 100 is used to generate RF energy for use during an ablation procedure. The RF generator 130 may include an RF source 132 that produces the RF energy and an RF generator component 134 that controls the timing, level, and/or other characteristics of the RF energy delivered by the RF generator 130. In embodiments, the RF generator 130 may be configured to deliver ablation energy to the ablation catheter 102 in a controlled manner in order to ablate the target tissue sites. Ablation of tissue within the heart is well known in the art, and thus for purposes of brevity, the RF generator 130 will not be described in further detail. Further details regarding RF generators are provided in U.S. Pat. No. 5,383,874, which is expressly incorporated herein by reference in its entirety for all purposes

In embodiments, the ablation energy delivered by the RF generator 130 may be altered by the control component 128 as described herein. For example, the control component 128 may send instructions to the RF generator component 134 to maintain a current level of ablation energy, increase the level of ablation energy and/or decrease the level of ablation energy based on the fluid flow condition determined by the fluid flow condition component 126.

For example, the RF generator component 134 may receive a signal from the control component 128 to provide a signal to the RF source 132 to decrease its energy output if the catheter is in a region with a lower fluid flow than an average fluid flow for a heart and/or a lower fluid flow than an average fluid flow of the portion of the heart that is near the tip assembly 104. In embodiments, the RF generator component 134 may receive instructions from the control component 128 to provide a signal to the RF source 132 to decrease its output if the catheter is moving from a region with a higher fluid flow to a region with a lower fluid flow. The RF generator component 134 may receive instructions from the control component 128 to provide a signal to the RF source 132 to decrease its output if the catheter is in a region with a fluid flow below a threshold fluid flow. In embodiments, the fluid flow and threshold fluid flow may be a fluid flow level (e.g., stagnant flow level, low flow level and/or high flow level) and/or a fluid flow rate.

According to embodiments, the RF generator component 134 may receive instructions from the control component 128 to provide a signal to the RF source 132 to increase its output if the catheter is a region with a higher fluid flow than an average fluid flow for a heart. In addition or alternatively, the RF generator component 134 may receive instructions from the control component 128 to provide a signal to the RF source 132 to increase its output if the catheter is moving from a region with a lower fluid flow to a region with a higher fluid flow. The RF generator component 134 may provide a signal to the RF source 132 to increase its output if the catheter is in a region with a fluid flow that is above a threshold fluid flow. In embodiments, the fluid flow and threshold fluid flow may be a fluid flow level (e.g., stagnant flow level, low flow level and/or high flow level), a fluid flow rate, a fluid flow velocity, a fluid flow acceleration (with or without a direction) and/or the like.

The illustrated system 100 also includes an irrigation system 136. The irrigation system 136 includes an irrigation fluid source 138 for providing cooling fluid, such as a saline, through the catheter 102 and out through the irrigation ports 108. In embodiments, the irrigation fluid source 138 may include a fluid reservoir and a pump to provide cooling fluid through the catheter. The irrigation system 136 also includes an irrigation fluid output component 140. The irrigation fluid output component 140 controls the timing, level, and/or other characteristics of the irrigation fluid provided by the irrigation system 136.

In embodiments, the control component 128 may receive the fluid flow condition determined by the fluid flow condition component 126 and send instructions to the irrigation fluid output component 140 to control irrigation system 136 based on the determined fluid flow condition. That is, in embodiments, the control component 128 may send instructions to the irrigation fluid output component 140 to provide a signal to the irrigation fluid source 138 to either increase or decrease the output of the irrigation system 136 based on a determined fluid flow condition.

For example, the control component 128 may send instructions to the irrigation fluid output component 140 to provide a signal to the irrigation fluid source 136 to decrease its output if the catheter is a region with a higher fluid flow than normal. In addition or alternatively, the control component 128 may send instructions to the irrigation fluid output component 140 to provide a signal to the irrigation fluid source 136 to decrease its output if the catheter is moving from a region with a lower fluid flow to a region with a higher fluid flow. The control component 128 may send instructions to the irrigation fluid output component 140 to provide a signal to the irrigation fluid source 136 to increase its output if the catheter is in a region with a fluid flow below a threshold fluid flow. In embodiments, the fluid flow and threshold fluid flow may be a fluid flow level (e.g., stagnant flow level, low flow level and/or high flow level), a fluid flow rate, a fluid flow velocity, a fluid flow acceleration (with or without a direction) and/or the like

According to embodiments, the control component 128 may send instructions to the irrigation fluid output component 140 to provide a signal to the irrigation fluid source 136 to increase its output if the catheter is a region with a lower fluid flow than normal. In addition or alternatively, the control component 128 may send instructions to the irrigation fluid output component 140 to provide a signal to the irrigation fluid source 136 to increase its output if the catheter is moving from a region with a higher fluid flow to a region with a lower fluid flow. The control component 128 may send instructions to the irrigation fluid output component 140 to provide a signal to the irrigation fluid source 136 to increase its output if the catheter is in a region with a fluid flow that is below a threshold fluid flow. In embodiments, the fluid flow and threshold fluid flow may be a fluid flow level (e.g., stagnant flow level, low flow level and/or high flow level), a fluid flow rate, a fluid flow velocity, a fluid flow acceleration (with or without a direction) and/or the like.

In embodiments, the irrigation fluid output component 140 may provide instructions to the fluid flow condition component 126. The instructions may indicate an amount by which the irrigation fluid output component 140 is signaling to the irrigation fluid source 138 to increase or decrease the output of the irrigation fluid source 138. As such, the fluid flow condition component 126 can consider the amount of irrigation fluid that is being output of the irrigation port(s) 108 when calculating the fluid flow condition, in order to distinguish between the blood flow component and the irrigation flow component of the fluid flow condition.

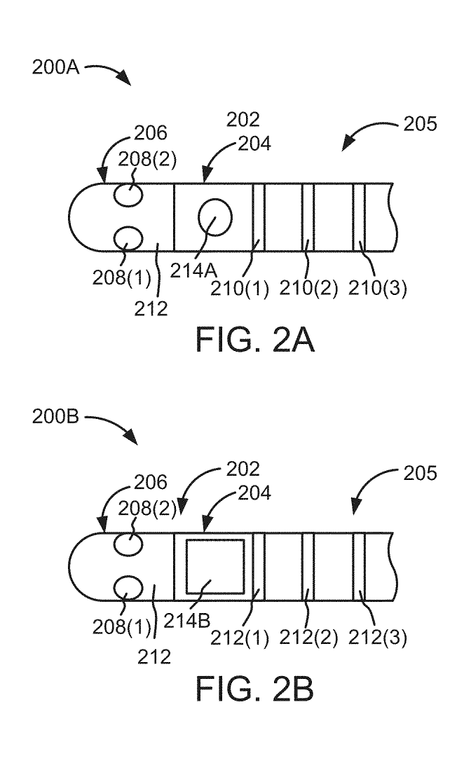

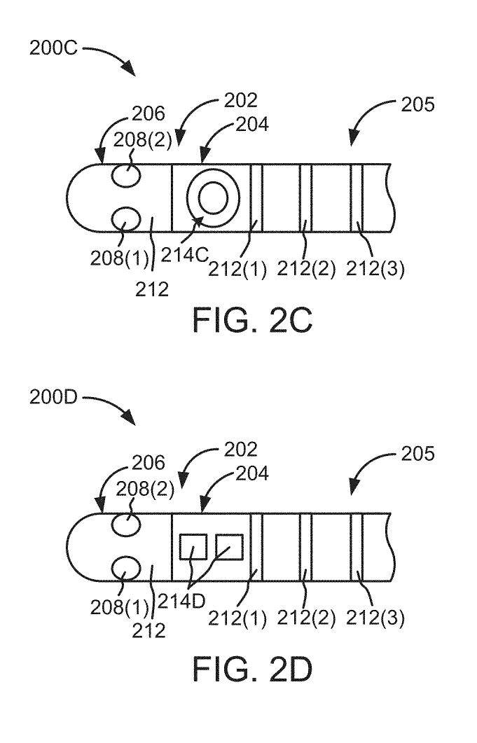

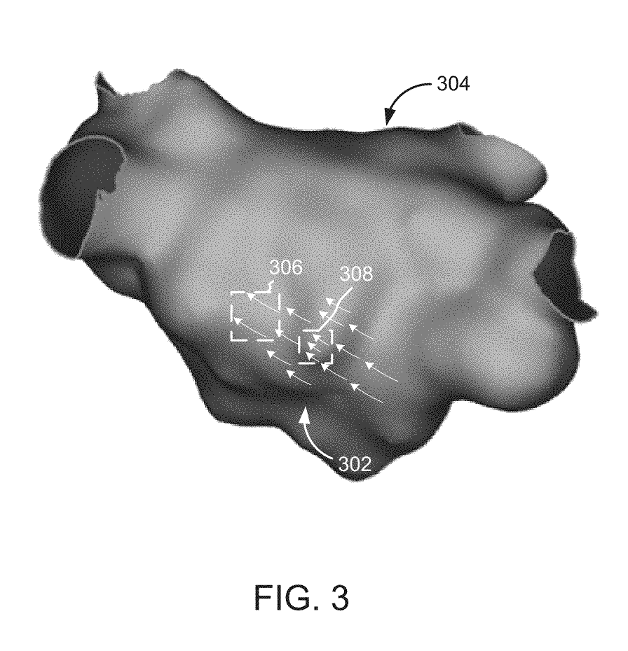

As mentioned above, the system 100 may include a mapping component 142 that receives signals from the electrodes 106. In embodiments, the mapping component 142 may be configured to detect, process, and record electrical signals associated with myocardial tissue via the electrodes 106. In embodiments, based on these electrical signals, a physician can identify the specific target tissue sites within the heart for ablative treatment. The mapping component 134 is configured to process signals from the electrodes 106 (and/or ring electrodes 212(1), 212(2), 212(3) depicted in FIG. 2), and generate an output to a display device 144. The display device 144 may be configured to present an indication of a tissue condition, effectiveness of an ablation procedure, fluid flow condition of fluid located near the tip assembly 104 (as illustrated in FIG. 3), irrigation fluid flow condition and/or the like (e.g., for use by a physician). In some embodiments, the display device 144 may include electrocardiogram (ECG) information, which may be analyzed by a user to determine the existence and/or location of arrhythmia substrates within the heart and/or determine the location of the tip assembly 104 within the heart. In embodiments, the output from the mapping component 142 can be used to provide, via the display device 144, an indication to the clinician about a characteristic of the tip assembly 104 and/or the tissue being mapped.

In instances where an output is generated to a display device 144 and/or other instances, the mapping component 142 may be operatively coupled to or otherwise in communication with the display device 144. In embodiments, the display device 144 may present various static and/or dynamic representations of information related to the use of the mapping and ablation system 100. For example, the display device 144 may present an image representing the target area, an image representing the catheter, an image representing a fluid flow condition (as illustrated in FIG. 3), an image representing an irrigation fluid flow condition, notifications relating to the fluid flow condition and/or irrigation fluid flow condition, and/or information related to EGMs, which may be analyzed by the user and/or by a processor of the RF ablation system to determine the existence and/or location of arrhythmia substrates within the heart, to determine the location of the catheter within the heart, and/or to make other determinations relating to use of the catheter and/or other catheters.

In embodiments, the display device 144 may be an indicator. Additionally or alternatively, the catheter handle assembly 114 may include a sensory output device 146 (e.g., a light, a speaker, a haptic device and/or the like) that is an indicator. In embodiments, whether the display device 144 and/or the sensory output device 146 is the indicator, the indicator may be capable of providing an indication related to a feature of the output signals received from the electrodes 106 and the sensors 110. For example, an indication to the clinician about a characteristic of the catheter, an indication to the clinician about a fluid flow rate and/or irrigation fluid flow rate, and/or an indication of the myocardial tissue interacted with and/or being mapped may be provided on the display device 144 and/or the sensory output device 146. In some cases, the indicator may provide a visual, audible and/or haptic indication to provide information concerning the characteristic of the catheter, the fluid flow rate, the irrigation fluid flow rate and/or the myocardial tissue interacted with and/or being mapped. In embodiments, the visual indication may take one or more forms. In some instances, a visual color, a sequence of visual colors, a light indication and/or a sequence of light indications may be provided on a display 144 and/or sensory output device 146. In embodiments, the visual color, the sequence of visual colors, the light indication and/or the sequence of light indications on a display 144 may be separate from or included on an imaged catheter on the display 144 if there is an imaged catheter. Such a color or light indicator may include a progression of lights or colors that may be associated with various levels of a characteristic proportional to the amplitude of the fluid flow rate. Alternatively, or in addition, an indicator indicating a level of a characteristic proportional to the amplitude or other characteristic of fluid flow, may be provided in any other manner on a display and/or with any audible or other sensory indication, as desired.

In embodiments, a visual indication may be an indication on a display device 144 (e.g., a computer monitor, touchscreen device, and/or the like) and/or a sensory output device 146 with one or more lights or other visual indicators. In one example of an indicator, a color of at least a portion of an electrode of a catheter imaged on a screen of the display 144 and/or a colored light emitted from the sensory output device 146 may change from a first color (e.g., red or any other color) when there is poor contact between the catheter and tissue to a second color (e.g., green or any other color different than the first color) when there is good contact between the catheter and the tissue and/or when ablation may be initiated after establishing good contact. Additionally or alternatively in another example of an indicator, when the amplitude and/or frequency spectrum of the EGM stops changing and/or reaches a lesion maturation amplitude or frequency spectrum threshold, a depicted color of an electrode on the imaged catheter, displayed on the display device 144 and/or a colored light emitted from the sensory output device 146 may change colors to indicate a level of lesion maturation. In a similar manner, an indicator may be utilized to indicate a viability of tissue to be ablated. In a similar manner, an indicator may be utilized to indicate if a fluid flow is below a threshold fluid flow. In the examples above, the changing color/light or changing other indicator (e.g., a number, an image, a design, etc.) may be located at a position on the display other than on the imaged catheter, as desired. According to embodiments, indicators may provide any type of information to a user. For example, the indicators discussed herein may be pass or fail type indicators showing when a condition is present or is not present and/or may be progressive indicators showing the progression from a first level to a next level of a characteristic (e.g., a fluid flow level and/or fluid flow rate).

In addition or alternatively, the system 100 may include speakers (not shown) and an audio sound and/or notification may be emitted from the speakers when a condition is present or is not present and/or may be progressive indicators showing the progression from a first level to a next level of a characteristic (e.g., a fluid flow level and/or fluid flow rate).

According to embodiments, the various components 126, 128, 134, 140, 142 included in the system 100 may be incorporated into one or more computing devices, e.g., the computing device 146. The computing device 146 may be, be similar to, include, or be included in type of computing device suitable for implementing embodiments of the disclosure. Examples of computing devices include specialized computing devices or general-purpose computing devices such “workstations,” “servers,” “laptops,” “desktops,” “tablet computers,” “hand-held devices,” and the like, all of which are contemplated within the scope of FIG. 1 with reference to various components of the system 100.

In embodiments, the computing device 146 includes a bus that, directly and/or indirectly, couples the following devices: a processing unit, a memory, an input/output (I/O) port, an I/O component, and a power supply. In embodiments, the components 126, 128, 134, 140, 142 are saved on the memory of the computing device 146 as instruction sets. In embodiments, the memory of the computing device 146 includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; or any other medium that can be used to store information and can be accessed by a computing device such as, for example, quantum state memory, and the like.

In embodiments, the memory of the computing device 146 stores computer-executable instructions for causing the processing unit of the computing device 146 to implement aspects of embodiments of system components 126, 128, 134, 140, 142 and/or to perform aspects of embodiments of methods and procedures discussed herein. Computer-executable instructions may include, for example, computer code, machine-useable instructions, and the like such as, for example, program components capable of being executed by one or more processors associated with a computing device. Program components 126, 128, 134, 140, 142 may be programmed using any number of different programming environments, including various languages, development kits, frameworks, and/or the like. Some or all of the functionality contemplated herein may also be implemented in hardware and/or firmware.

Any number of additional components, different components, and/or combinations of components may also be included in the computing device 146. The bus represents what may be one or more busses (such as, for example, an address bus, data bus, or combination thereof). Similarly, in embodiments, the computing device 146 may include a number of processing units (which may include, for example, hardware, firmware, and/or software computer processors), a number of memory components, a number of I/O ports, a number of I/O components, and/or a number of power supplies. Additionally any number of these components, or combinations thereof, may be distributed and/or duplicated across a number of computing devices.

FIGS. 2A-2D depict illustrative catheter tip assemblies, in accordance with embodiments of the disclosure. Referring to FIG. 2A, the illustrated catheter 200A includes a tip assembly 202 coupled to a distal end of a catheter body 205, having a tip body 204, and an ablation electrode 206 used to perform mapping and ablation functions. In embodiments, the ablation functions may be performed, in part, by the ablation electrode 206, which may function as an RF electrode. The mapping functions may be performed, at least in part, by mapping electrodes 208 and mapping ring electrodes 210.

The illustrated tip assembly 202 includes a generally hollow ablation electrode 206 having an open interior region defined by an exterior wall 212 of the tip assembly 202. In the illustrated embodiments, the hollow tip body 204 has a generally cylindrical shape, but in other embodiments, the tip body 204 may have any number of different shapes such as, for example, an elliptical shape, a polygonal shape, and/or the like. By way of an example and not limitation, embodiments of the tip assembly 202 may have a diameter on the order of about 0.08-0.1 inches, a length on the order of about 0.2-0.3 inches, and an exterior wall 212 with a thickness on the order of about 0.003-0.004 inches. According to embodiments, the ablation electrode 206 may be formed from a conductive material. For example, some embodiments use a platinum-iridium alloy. Some embodiments use an alloy with approximately 90% platinum and 10% iridium. The conductive material of the ablation electrode 206 is used to conduct RF energy used to form legions during the ablation procedure.

The illustrated tip assembly 202 also includes a sensor 214A that senses signals indicative of a fluid flow condition. In embodiments, the sensor 214A may be, be similar to, include, or be included in, the sensor 110 discussed in FIG. 1. While only one sensor 214A is shown, in embodiments, there may be multiple sensors 214A included on the tip assembly 202. In embodiments, the sensor 214A may be different shapes. For example, the sensor 214A may be circular, as shown in FIG. 2A, the sensor 214B may be square, as shown in FIG. 2B, or have any other type of shape suitable for measuring a fluid flow condition. In embodiments, the sensor 214C, 214D included in the tip assembly 202 may include multiple parts, as shown in FIGS. 2C, 2D. For example, the sensor 214C, 214D may include an optical and/or ultrasonic sensor and receiver, a heat emitter and receiver and/or the like. In embodiments, the sensors 214A-214D may be one or more of the following types of sensors: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor and/or an ionic concentration sensor.

FIG. 3 depicts a representation of fluid flow conditions 302 on a representation of a cardiac structure 304, in accordance with embodiments of the disclosure. As discussed above, a sensor (e.g., the sensor 110 depicted in FIG. 1) located on the tip assembly (e.g., the tip assembly 104 depicted in FIG. 1) of a catheter (e.g., the catheter 102 depicted in FIG. 1) may sense signals that are indicative of one or more fluid flow conditions 302 of fluid located near the tip assembly. The sensor may transmit these signals to a fluid flow condition component (e.g., the fluid flow condition component 126 depicted in FIG. 1) for determining fluid flow conditions 302 from the signals. After the fluid flow condition component determines the fluid flow conditions 302 of the fluid located near the tip assembly, the fluid flow condition component may provide the fluid flow conditions 302 to a mapping component (e.g., the mapping component 142 depicted in FIG. 1). The mapping component may then cause a display device to display a representation of the fluid flow conditions 302 either alone or on a representation of a cardiac structure, such as the cardiac structure 304 depicted in FIG. 3.

In embodiments, the direction of the fluid flow conditions 302 may be represented by an arrow, such as the arrows depicted in FIG. 3. In the embodiment shown, the fluid flow conditions 302 are fluid flow rates. As such, in embodiments, the speed of the fluid flow conditions 302 may be correlated to the length of the arrow. For example, the speed of the fluid flow rates located in region 304 is greater than the speed of the fluid flow rates located in region 306. Based on this representation of the fluid flow conditions 302, a control component (e.g., the control component 128 depicted in FIG. 1) may control a RF generator component (e.g., the RF generator 134 component depicted in FIG. 1) and/or an irrigation fluid output component (e.g., the irrigation fluid output component 138 depicted in FIG. 1). For example, if the tip assembly repositions to ablate a portion of cardiac tissue located in region 308 after ablating a portion of cardiac tissue located in region 306, the control component may instruct the RF generator component to decrease the amount of RF energy emitted and/or may instruct the irrigation fluid output component to increase the irrigation output. In addition or alternatively, the control component may provide a notification to a user of the catheter that the tip assembly is repositioning into a region 308 that has a lower fluid flow condition 302 than the region 306 the tip assembly was previously located in and/or provide a notification that the fluid flow conditions 302 are below a fluid flow condition threshold if the region 308 includes fluid flow rates that are below a threshold fluid flow rate. In this manner, ineffective passive cooling, charring and steam popping, may be avoided.

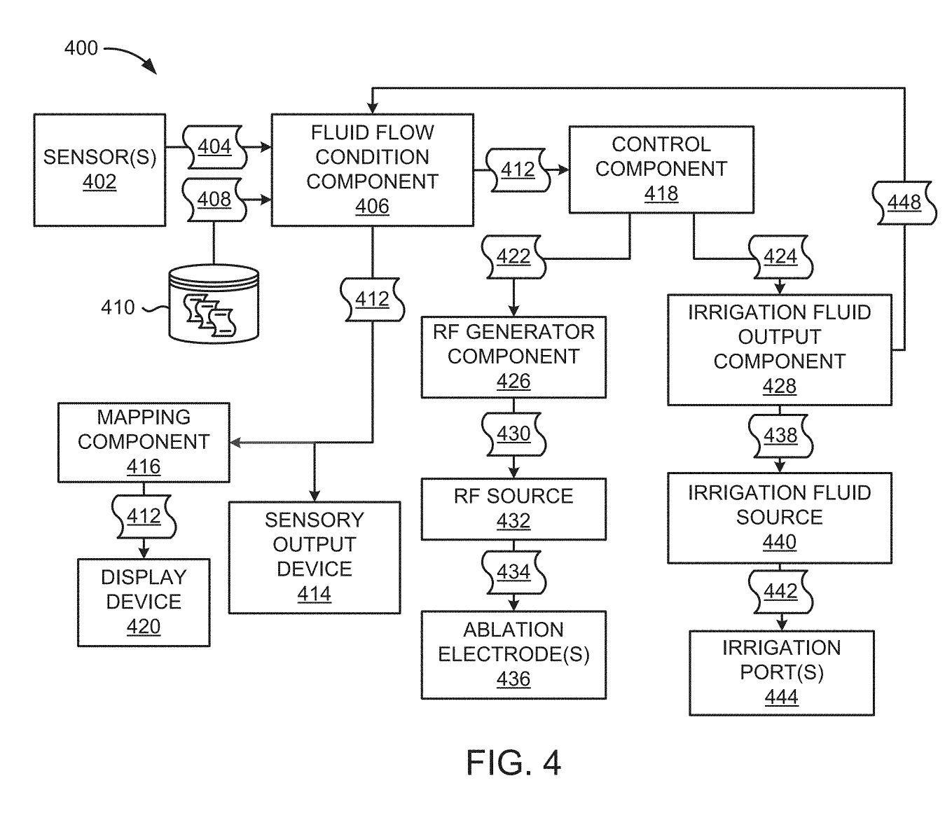

FIG. 4 is a schematic block diagram of an illustrative process 400 determining a fluid flow condition and providing an indication of the fluid flow condition and/or controlling a catheter based on the determined fluid flow condition, in accordance with embodiments of the disclosure. Because any number of the various components depicted in FIG. 4 may be implemented in any number of different combinations of devices (such as, e.g., aspects of embodiments of the system 100 depicted, in FIG. 1), FIG. 4 is depicted, and described, without regard to the particular device(s) within which each component is implemented, but is rather discussed in the context of system components and their functions.

As shown in FIG. 4, the process flow 400 includes a sensor 402 that senses signals indicative of fluid flow condition and provides the set 404 to a fluid flow condition component 406. In embodiments, the sensor may be, be similar to, include, or be included in, the sensor 110 and/or the sensors 214A-214D depicted in FIG. 1 and FIGS. 2A-2D and may be implemented, for example, in a catheter (e.g., the catheter 102 depicted in FIG. 1). The sensor 402 may be or include one or more of the following types of sensors: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor and/or an ionic concentration sensor.

In embodiments, the fluid flow condition component 406 analyzes the set 404 to determine one or more fluid flow conditions located near the sensor(s) 402 using a set of instructions 408, which may retrieved from a storage device 410. In embodiments, the fluid flow condition component 406 may be, be similar to, include, or be included in, the fluid flow condition component 126 depicted in FIG. 1, and may also, or alternatively, be implemented in a medical device (e.g., the medical device 146 depicted in FIG. 1). In embodiments, the set of instructions 408 for determining one or more fluid flow conditions may be one or more correlation tables which correlate the type of sensor 402 and signals that the senor 402 receives to a fluid flow condition. In embodiments, the fluid flow condition may be a fluid flow level (e.g., stagnant flow level, low flow level and/or high flow level), a fluid flow rate, a fluid flow velocity, a fluid flow acceleration (with or without a direction) and/or the like.

The storage device 410 may include computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the storage device 410 stores computer-executable instructions for causing a processor to implement aspects of embodiments of system components discussed herein and/or to perform aspects of embodiments of methods and procedures discussed herein.

The computer-executable instructions may include, for example, computer code, machine-useable instructions, and the like such as, for example, program components capable of being executed by one or more processors associated with the computing device. Program components may be programmed using any number of different programming environments, including various languages, development kits, frameworks, and/or the like. Some or all of the functionality contemplated herein may also, or alternatively, be implemented in hardware and/or firmware.

In embodiments, the fluid flow condition component 406 may output the one or more determined fluid flow conditions 412 to a sensory output device 414, a mapping component 416 and/or a control component 418. In embodiments, the sensory output device 414 may be, be similar to, include, or be included in, the sensory output device 146 depicted in FIG. 1. In embodiments, the mapping component 416 and the control component 418 may be, be similar to, include, or be included in, the mapping component 142 and/or the control component 128 depicted in FIG. 1, and may also, or alternatively, be implemented in a medical device (e.g., the medical device 146 depicted in FIG. 1).

In embodiments where the one or more fluid flow conditions 412 are provided to the mapping component 416, the mapping component may output the fluid flow conditions 412 to a display device 420 and cause the display device 420 to display a representation of the fluid flow conditions 412 (e.g., the fluid flow conditions 302 depicted in FIG. 3) either alone or on a representation of a cardiac structure (e.g., the cardiac structure 304 depicted in FIG. 3).

In embodiments where the one or more fluid flow conditions 412 are provided to the control component 418, the control component 418 may provide instructions 422, 424 to the RF generator component 426 and/or the irrigation fluid output component 428. In embodiments, the RF generator component 426 and the irrigation fluid output component 428 may be, be similar to, include, or be included in, the RF generator component 134 and/or the irrigation fluid output component 138 depicted in FIG. 1, and may also, or alternatively, be implemented in a medical device (e.g., the medical device 146 depicted in FIG. 1).

After receiving instructions from the control component 418, the RF generator component 426 may provide a signal 430 to an RF source 432 to either increase or decrease RF output. In embodiments, the RF source 432 may be, be similar to, include, or be included in, the RF source 132 depicted in FIG. 1, and may also, or alternatively, be implemented in a medical device (e.g., the medical device 146 depicted in FIG. 1).

In embodiments, the RF generator component 426 may provide a signal 430 to the RF source 432 to decrease its output if the catheter is a region with a lower fluid flow than normal. In addition or alternatively, the RF generator component 426 may provide a signal 430 to the RF source 432 to decrease its output if the catheter is moving from a region with a higher fluid flow to a region with a lower fluid flow. In addition or alternatively, the RF generator component 426 may provide a signal 430 to the RF source 432 to decrease its output if the catheter is in a region with a fluid flow below a threshold fluid flow. In embodiments, the fluid flow and the threshold fluid flow may be a fluid flow level (e.g., stagnant flow level, low flow level and/or high flow level), a fluid flow rate, a fluid flow velocity, a fluid flow acceleration (with or without a direction) and/or the like.

Alternatively, the RF generator component 426 may provide a signal 430 to the RF source 432 to increase its output if the catheter is a region with a higher fluid flow than normal. In addition or alternatively, the RF generator component 426 may provide a signal 430 to the RF source 432 to increase its output if the catheter is moving from a region with a lower fluid flow to a region with a higher fluid flow. In addition or alternatively, the RF generator component 426 may provide a signal 430 to the RF source 432 to increase its output if the catheter is in a region with a fluid flow that is above a threshold fluid flow. After the RF source 432 receives a signal 430 from the RF generator component 426, the RF source will output the RF energy 434, according to the signal 430, to the ablation electrodes 436. In embodiments, the ablation electrodes 436 may be, be similar to, include, or be included in, the ablation electrodes 105 depicted in FIG. 1, and may also, or alternatively, be implemented in a RF generator (e.g., the RF generator 130 depicted in FIG. 1).

In embodiments where the control component 416 provides instructions 424 to the irrigation fluid output component 428, the irrigation fluid output component 428 may provide a signal 438 to an irrigation fluid source 440 to either increase or decrease the irrigation fluid output 442 to the irrigation port(s) 444. In embodiments, the irrigation fluid output component 428 may provide a signal 438 to the irrigation fluid source 440 to decrease its output if the catheter is a region with a higher fluid flow than normal. In addition or alternatively, the irrigation fluid output component 428 may provide a signal 440 to the irrigation fluid source 440 to decrease its output 442 if the catheter is moving from a region with a lower fluid flow to a region with a higher fluid flow. The irrigation fluid output component 428 may provide a signal 438 to the irrigation fluid source 440 to increase its output 442 if the catheter is in a region with a fluid flow below a threshold fluid flow.

According to embodiments, the irrigation fluid output component 428 may provide a signal 438 to the irrigation fluid source 440 to increase its output 442 if the catheter is a region with a lower fluid flow than normal. In addition or alternatively, the irrigation fluid output component 428 may provide a signal 438 to the irrigation fluid source 440 to increase its output 442 if the catheter is moving from a region with a higher fluid flow to a region with a lower fluid flow. In addition or alternatively, the irrigation fluid output component 428 may provide a signal 438 to the irrigation fluid source 440 to increase its output 442 if the catheter is in a region with a fluid flow that is below a threshold fluid flow. In embodiments, the fluid flow may be a fluid flow level (e.g., stagnant flow level, low flow level and/or high flow level), a fluid flow rate, a fluid flow velocity, a fluid flow acceleration (with or without a direction) and/or the like.

In embodiments, the irrigation fluid output component 428 may provide instructions 448 to the fluid flow condition component 406. The instructions 448 may indicate an amount by which the irrigation fluid output component 424 is signaling to the irrigation fluid source 440 to increase or decrease the irrigation fluid source's output 442. As such, the fluid flow condition component 406 can consider the amount of irrigation fluid that is being output of the irrigation port(s) 444 when calculating the fluid flow condition, in order to distinguish between the blood flow component and the irrigation flow component of the fluid flow condition.

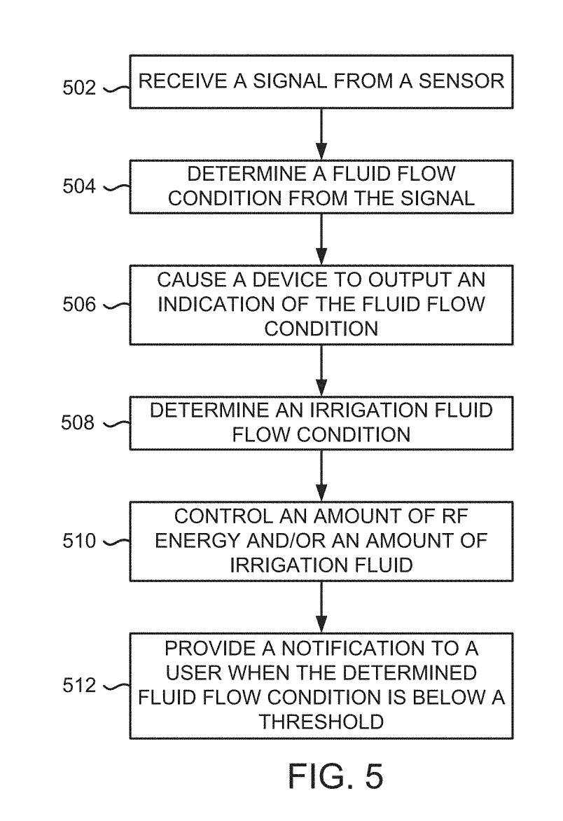

FIG. 5 is a flow diagram depicting an illustrative method 500 for determining a fluid flow condition and providing an indication of the fluid flow condition and/or controlling a catheter based on the determined fluid flow condition, in accordance with embodiments of the disclosure, in accordance with embodiments of the disclosure. Embodiments of the method 500 may be performed by one or more components of a medical system such as, for example, the medical system 100 depicted in FIG. 1, using a process such as, for example, the illustrative process 400 depicted in FIG. 4.

Embodiments of the illustrative method 500 include receiving a signal from a sensor (block 502). The sensing may be performed by a sensor (e.g., the sensor 110 depicted in FIG. 1 and/or the sensors 214A-214D depicted in FIG. 2). The parameter that is sensed by the sensor may include any number of different types of information such as, for example, temperature(s), pressure(s), reflected optical and/or audio signal(s), pH(s), ionic concentration of other element(s), luminescence(s) absorbed in oxygen, mechanical parameter(s) (e.g., a strain gauge deflection) and/or the like discussed above in relation to FIG. 1.

In embodiments, the method 500 may further include determining a fluid flow condition from the received signal(s) (block 504). Determining a fluid flow condition may be performed by a fluid flow condition component (e.g., the fluid flow condition component 128 depicted in FIG. 1 and/or the fluid flow condition component 406 depicted in FIG. 4). In embodiments, the method 500 may further include causing a device to output an indication of the fluid flow condition (block 506). Causing a device to output an indication of the fluid flow condition. In embodiments, the indication may be, be similar to, include, or be included in the indicator described in relation to FIG. 1 above and the device may be, be similar to, include, or be included in the display device 144 and/or the sensory output device 146 depicted in FIG. 1 above. For example, in embodiments, causing a device to output an indication of the fluid flow condition may include causing a display device (e.g., the display device 144 depicted in FIG. 1 and/or the display device 416 depicted in FIG. 4) to display a representation of the fluid flow condition (e.g., the representation of the fluid flow condition 302 depicted in FIG. 3). In embodiments, the fluid flow condition may be represented on a representation of a cardiac structure (e.g., the cardiac structure 304 depicted in FIG. 3).

In embodiments, method 500 may include determining an irrigation fluid flow condition 506 (block 508). An irrigation fluid flow condition may be determined by a fluid flow condition component (e.g., the fluid flow condition component 128 depicted in FIG. 1 and/or the fluid flow condition component 406 depicted in FIG. 4).

In embodiments, the method 500 may further include controlling an amount of RF energy and/or an amount of irrigation fluid (block 510). In embodiments, controlling an amount of RF energy and/or an amount of irrigation fluid may be performed by a control component (e.g., the control component 128 depicted in FIG. 1 and/or the control component 416 depicted in FIG. 4) communicating to an RF generator component (e.g., the RF generator component 134 depicted in FIG. 1 and/or the RF generator component 422 depicted in FIG. 4) and an irrigation fluid output component (e.g., the irrigation fluid output component 140 depicted in FIG. 1 and/or the irrigation fluid output component 424 depicted in FIG. 4).

In embodiments, the method 500 may further include providing a notification to a user when the determined fluid flow condition is below a threshold (block 512). In embodiments, the notification may be provided as an indication (e.g., the indicators discussed above in reference to FIG. 1).

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter system comprising:
a catheter comprising a tip assembly, the tip assembly including at least one sensor; and
a processing device configured to:
receive a signal from the at least one sensor, the signal being indicative of a fluid flow condition of fluid located near the tip assembly;
determine the fluid flow condition using the signal; and
cause a device to output an indication of the determined fluid flow condition.

2. The system of claim 1, wherein the fluid flow condition includes at least one of: a stagnant flow condition, a low flow condition and a high flow condition.

3. The system of claim 1, wherein the fluid flow condition includes a fluid flow rate.

4. The system of claim 3, the processing device being further configured to provide a notification to a user when the determined fluid flow rate is below a fluid flow rate threshold.

5. The system of claim 3, the processing device being further configured to cause the display device to display a representation of the determined fluid flow rate.

6. The system of claim 4, the processing device being further configured to cause the display device to display the representation of the determined fluid flow rate on a representation of a cardiac structure.

7. The system of claim 1, wherein the device is a sensory output device incorporated into a handle of the catheter and wherein the indication of the determined fluid flow condition is represented by at least one of: one or more colors of light, one or more sequences of lights, one or more sounds, one or more sequence of sounds and one or more haptics.

8. The system of claim 1, wherein the tip assembly includes a conductive exterior wall for delivering RF energy for an RF ablation procedure and an irrigation port, the processing device being further configured to control at least one of: an amount of irrigation fluid provided to the irrigation port and an amount of RF energy delivered by the conductive exterior wall, based on the determined fluid flow condition.

9. The system of claim 8, wherein the fluid flow condition includes a fluid flow rate and wherein to control an amount of irrigation fluid provided to the irrigation port based on the determined fluid flow rate, the processing device is configured to increase the amount of irrigation fluid provided to an irrigation port when the determined fluid flow rate is below a fluid flow rate threshold.

10. The system of claim 1, wherein the catheter is an irrigated catheter, the processing device being further configured to determine an irrigation flow condition of irrigation fluid using the determined fluid flow condition.

11. The system of claim 1, the at least one flow sensor including at least one of the following: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor and an ionic concentration sensor.

12. A method comprising:
receiving a signal from at least one sensor located on a tip assembly of a radio frequency (RF) ablation catheter, the signal being indicative of a fluid flow condition of fluid located near the tip assembly;
determining the fluid flow condition using the signal; and
dynamically adjusting, based on the determined fluid flow rate, at least one of an amount of RF energy delivered by the tip assembly and an amount of irrigation fluid provided to an irrigation port included in the tip assembly.

13. The method of claim 12, further comprising determining an irrigation flow rate using the determined fluid flow rate and providing a notification to a user when the determined fluid flow rate is below a fluid flow rate threshold.

14. The method of claim 12, further comprising causing a display device to display a representation of the determined fluid flow rate.

15. The method of claim 12, further comprising:
displaying, using a display device, a representation of a cardiac structure; and
displaying a representation of the determined fluid flow rate on the representation of the cardiac structure.

16. The method of claim 12, the at least one flow sensor including at least one of the following: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor and an ionic concentration sensor.

17. A catheter system comprising:
a catheter comprising a tip assembly, the tip assembly including: a conductive exterior wall for delivering radio frequency (RF) energy for an RF ablation procedure, an irrigation port and at least one sensor; and
a processing device configured to:
receive a signal from the at least one sensor, the signal being indicative of a fluid flow rate of fluid located near the tip assembly;
determine the fluid flow rate using the signal; and
control, based on the determined fluid flow rate, at least one of an amount of delivered RF energy and an amount of irrigation fluid provided to the irrigation port.

18. The system of claim 17, the processing device being further configured to cause the display device to display the representation of the determined fluid flow rate on a representation of a cardiac structure.

19. The system of claim 17, the processing device being further configured to determine an irrigation flow rate of irrigation fluid exiting the irrigation port using the determined fluid flow rate.

20. The system of claim 17, the at least one flow sensor including at least one of the following: a volumetric fluid flow sensor, a linear pair fluid flow sensor, a concentric pair fluid flow sensor, a pressure sensor, an oxygen sensor, an optical sensor, an ultrasonic sensor, a mechanical sensor, a thermal sensor, a pH sensor and an ionic concentration sensor.
